(19) United States
(12) Patent Application Publication
(10) Pub. No.: US 2026/0102330 A1
(43) Pub. Date: Apr. 16, 2026

(54) FERMENTED PARSLEY COMPOSITIONS FOR TREATMENT OF INFLAMMATION

(71) Applicant: Agency Eleven LLC, New York, NY (US)

(72) Inventors: Carly Guerra, New York, NY (US); Chantao Wang, Sarasota, FL (US); Michael Ingrassia, Hauppauge, NY (US); Paul Stettnisch, Lindenhurst, NY (US)

(73) Assignee: Agency Eleven LLC, New York, NY (US)

(21) Appl. No.: 18/913,408

(22) Filed: Oct. 11, 2024

(51) Int. Cl. A61K 8/9789; A61K 8/34; A61K 8/35; A61K 8/9728; A61Q 19/00

(52) U.S. Cl. CPC A61K 8/9789; A61K 8/345; A61K 8/35; A61K 8/9728; A61Q 19/00; A61K 2800/805; A61K 2800/85

(57) ABSTRACT

In an embodiment, a cosmetic composition for treating inflammation is disclosed. The composition includes an extract of Carum Petroselinum (parsley), Saccharomyces Ferment Filtrate, and 1,2 Hexanediol, Hydroxyacetophenone. The composition is for topical administration. The composition may be administered at a 2.5, 5, or 10% concentration. The composition includes 0.5-2.0% parsley extract, 96.0-99.0% Saccharomyces Ferment Filtrate, 0.5-2.0% Hydroxyacetophenone, and 0.2-0.6% 1,2 Hexanediol by weight. The composition may be administered neat as a water-based composition. The composition may be combined with one or more oils and administered as a broth.

### COPYRIGHT NOTICE

A portion of the disclosure of this patent document contains material, which is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### BACKGROUND

This application relates to the field of cosmetics, and in particular, the anti-inflammatory effects of parsley ferment compositions in human keratinocytes. Human keratinocytes are the most common type of cell in the epidermis, the outermost layer of the skin, and make up about 90% of epidermal cells.

The beauty and personal care industry is expected to reach close to $736 billion in global revenue by 2028. In a world where people are more health-conscious than ever, it is no surprise that customers are becoming increasingly aware of the need for natural skin care products. Natural products can be gentler on the skin and are less likely to irritate it or cause dryness or breakouts. Additionally, because these products are free of harsh chemicals and preservatives, they also tend to have fewer side effects and are better for the environment. Moreover, natural products often contain ingredients like essential oils and herbs which can help nourish the skin with vitamins, minerals, and antioxidants that can help reduce inflammation and signs of aging.

Parsley contains anti-inflammatory and antioxidant properties, as well as vitamins and minerals that can benefit skin health. For example, its anti-inflammatory and antibacterial qualities may aid in the treatment and prevention of acne. Furthermore, the antioxidants present in parsley may aid in slowing the aging process and protecting the skin from oxidative stress. As customers are turning to natural alternatives to traditional beauty products laden with artificial chemicals, there is a need for improvement upon methods of using parsley in skincare products.

### SUMMARY

The present invention comprises a cosmetic composition for treating inflammation. According to an embodiment, the composition comprises an extract of Carum Petroselinum (parsley), Saccharomyces Ferment Filtrate, and 1,2 Hexanediol, Hydroxyacetophenone. The composition is for topical administration and may be in the form of a lotion, cream, or gel. The composition may be administered at a 2.5, 5, or 10% concentration. The composition comprises 0.5-2.0% parsley extract, 96.0-99.0% Saccharomyces Ferment Filtrate, 0.5-2.0% Hydroxyacetophenone, and 0.2-0.6% 1,2 Hexanediol by weight. The composition may be administered neat as a water-based composition. The composition may be combined with one or more oils and administered as a broth.

In another embodiment, a method of treating inflammation is disclosed. The method comprises administering to an individual in need of treatment a cosmetic composition comprising an extract of Carum Petroselinum (parsley), Saccharomyces Ferment Filtrate, and 1,2 Hexanediol, Hydroxyacetophenone. The composition may be administered topically at a 2.5, 5, or 10% concentration. The composition comprises 0.5-2.0% parsley extract, 96.0-99.0% Saccharomyces Ferment Filtrate, 0.5-2.0% Hydroxyacetophenone, and 0.2-0.6% 1,2 Hexanediol by weight. The method may comprise administering the composition neat as a water-based composition. The method may comprise combining the composition with one or more oils and administering it as a broth. The composition may be in the form of a lotion, cream, or gel.

In another embodiment, a method of manufacturing a cosmetic composition for treating inflammation is disclosed. The method comprises combining an extract of Carum Petroselinum (parsley) with water and yeast. The method further comprises fermenting the combined ingredients, inactivating and decolorizing the fermented ingredients, and subjecting the composition to centrifugal filtration. The method further comprises adding 1,2 Hexanediol, Hydroxyacetophenone to the composition and subjecting the composition to at least one round of sterilization. According to an embodiment, the yeast comprises Saccharomyces. The combined ingredients may be fermented at 28° C. for 72 hours. The composition may comprise 0.5-2.0% parsley extract, 96.0-99.0% Saccharomyces Ferment Filtrate, 0.5-2.0% Hydroxyacetophenone, and 0.2-0.6% 1,2 Hexanediol by weight. The composition may be manufactured for topical administration in the form of a lotion, cream, or gel.

In another embodiment, a method for repairing inflammation damage in human keratinocytes is disclosed. The method comprises applying a composition comprising a Saccharomyces fermented Carum Petroselinum (parsley) extract to the keratinocytes. The composition may comprise 0.5-2.0% parsley extract, 96.0-99.0% Saccharomyces Ferment Filtrate, 0.5-2.0% Hydroxyacetophenone, and 0.2-0.6% 1,2 Hexanediol by weight.

The foregoing summary is illustrative only and is not intended to be in any way limiting. In addition to the illustrative aspects, embodiments, and features described above, further aspects, embodiments, and features will become apparent by reference to the following detailed description.

### BRIEF DESCRIPTION OF THE DRAWINGS

The invention is illustrated in the figures of the accompanying drawings which are meant to be exemplary and not limiting, in which like references are intended to refer to like or corresponding parts.

FIG. 1 is a table illustrating product specifications of a parsley ferment composition according to embodiments of the present invention.

FIG. 2 is a flow diagram of an example process for manufacturing a parsley ferment composition according to embodiments of the present invention.

FIG. 3 is a bar graph illustrating the effects of example parsley ferments on HaCaT cell viability according to embodiments of the present invention.

FIG. 4 is a bar graph illustrating parsley ferment mediated inhibition of Interleukin 8 release from HaCaT cells according to embodiments of the present invention.

FIG. 5 is a bar graph illustrating parsley ferment mediated inhibition of Interleukin 6 release from HaCaT cells according to embodiments of the present invention.

### DETAILED DESCRIPTION OF THE INVENTION

Subject matter will now be described more fully hereinafter with reference to the accompanying drawings, which form a part hereof, and which show, by way of illustration, exemplary embodiments in which the invention may be practiced. Subject matter may, however, be embodied in a variety of different forms and, therefore, covered or claimed subject matter is intended to be construed as not being limited to any example embodiments set forth herein; example embodiments are provided merely to be illustrative. It is to be understood that other embodiments may be utilized and structural changes may be made without departing from the scope of the illustrative embodiments. Likewise, a reasonably broad scope for claimed or covered subject matter is intended. Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of exemplary embodiments in whole or in part. Among other things, for example, subject matter may be embodied as methods or compositions. The following detailed description is, therefore, not intended to be taken in a limiting sense.

The present disclosure provides compositions and methods for treatment of inflammation using fermented parsley. Customers are becoming increasingly aware of the need for natural skin care products that are gentler on the skin and are less likely to cause irritation, dryness or breakouts than products containing artificial chemicals. Because natural products are free of harsh chemicals and preservatives, they also tend to have fewer side effects and are better for the environment.

The disclosed compositions comprise therapeutically effective doses of parsley ferment for topical administration or application to the skin. The methods comprise administering topically to an individual in need of treatment a composition comprising a therapeutically effective amount of parsley ferment. The composition may comprise a Saccharomyces fermented Carum Petroselinum (parsley) extract which is applied to human keratinocytes to repair inflammation damage. A therapeutically effective amount may be delivered in one or more doses in the form of a lotion, cream, or gel.

The term “treatment” as used herein refers to reduction in one or more symptoms or features associated with the presence of the particular condition being treated. Treatment does not necessarily mean complete cure or remission, nor does it preclude recurrence or relapses. For example, treatment in the present disclosure means reducing one or more symptoms associated with inflammation. Treatment can be orientated symptomatically, for example, to suppress symptoms. It can be effected over a short period, over a medium term, or can be a long-term treatment, such as, for example, within the context of a maintenance therapy. Administrations may be intermittent, periodic, or continuous.

The term “therapeutically effective amount” as used herein in reference to a single agent is the amount sufficient to achieve, in a single or multiple doses, the intended purpose of treatment. The exact amount desired or required will vary depending on the particular compound or composition used, its mode of administration, patient specifics, and the like. Appropriate effective amounts can be determined by one of ordinary skill in the art informed by the present disclosure.

Where a range of values is provided in this disclosure, it should be understood each intervening value to the tenth of the value of the lower limit, and all intervening ranges between the upper and lower limit of that range are encompassed within the invention, unless clearly indicated otherwise. Furthermore, a reference to singular form also includes its plural form, and vice versa.

The present compositions expand on the benefits obtained by using simple parsley extractions in skincare. Sophisticated fermentation methods untap the benefits of this botanical herb to yield results that surpass traditional extraction methods. As discussed herein, fermenting a parsley extract by a strain of Saccharomyces enhances its anti-inflammatory functionality. According to an embodiment, the disclosed parsley ferment compositions may be used for calming, soothing, anti-itching and anti-irritation functions, rosacea applications, abnormal pigmentation prevention, as after-sun and anti-aging products, and for other cosmetic uses.

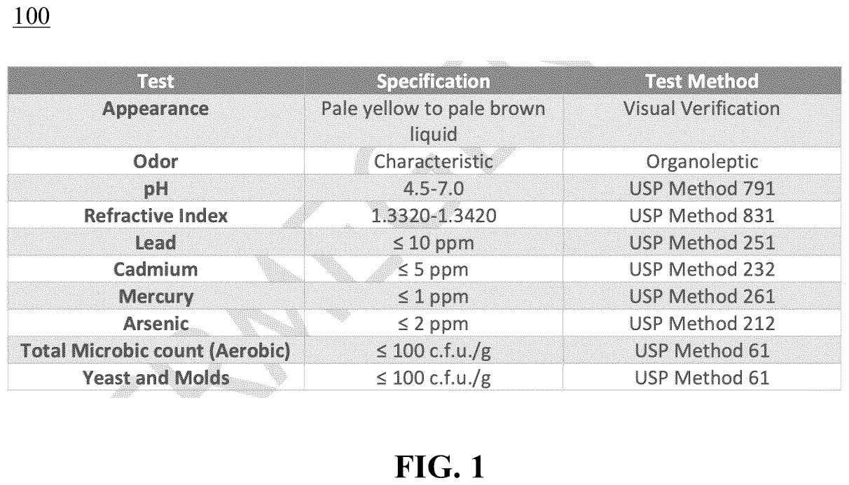

According to an embodiment, the International Nomenclature Cosmetic Ingredient name of the disclosed parsley ferment comprises Saccharomyces Ferment Filtrate, Carum Petroselinum (Parsley) Extract, 1,2 Hexanediol, Hydroxyacetophenone. The parsley ferment is miscible with aqueous phases, preferably added after the emulsion has been created. If properly stored in tightly closed containers in a dry, cool place away from light and heat, the parsley ferment may have a shelf life of two years. In an embodiment, Carum Petroselinum Extract comprises 0.5-2.0% of the parsley ferment composition by weight. The disclosed composition further comprises 96.0-99.0% Saccharomyces Ferment Filtrate, 0.5-2.0% Hydroxyacetophenone, and 0.2-0.6% 1,2 Hexanediol by weight. Further product specification details are disclosed in FIG. 1.

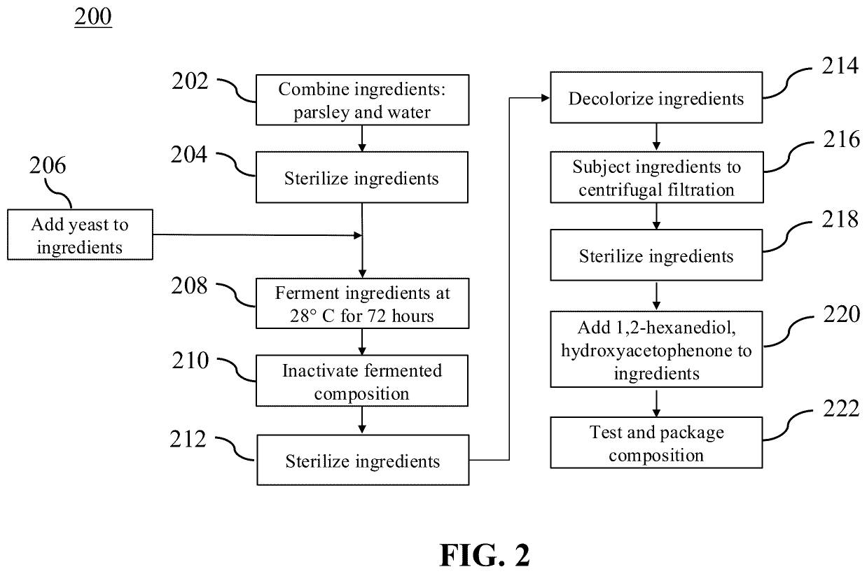

With reference to FIG. 2, a process 200 of manufacturing a parsley ferment composition in accordance with some embodiments will now be described. The process of FIG. 2 comprises steps 202 through 222.

At steps 202 and 204, an extract of parsley is mixed with water and the composition is sterilized. At steps 206 and 208, yeast such as Saccharomyces is added to the sterilized composition which is fermented at 25-31° C. for 65-80 hours. According to an embodiment, the composition is fermented at 28° C. for 72 hours. At step 210, the fermented composition is inactivated using at least one inactivation technique. The inactivated composition is then sterilized (step 212), decolorized (step 214), subjected to centrifugal filtration (step 216), and sterilized again (step 218). At step 220, 1,2 Hexanediol, Hydroxyacetophenone is added to the composition. In an embodiment, the composition comprises 0.5-2.0% parsley extract, 96.0-99.0% Saccharomyces Ferment Filtrate, 0.5-2.0% Hydroxyacetophenone, and 0.2-0.6% 1,2 Hexanediol by weight. The composition is now ready for packaging and testing (step 222).

### EXAMPLES

The following non-restrictive examples describe experimental uses of the present formulations to treat inflammation. According to an example embodiment, an extract of parsley was fermented by a strain of Saccharomyces and evaluated in a standard cell based anti-inflammatory model system. It was observed that the fermented parsley was well tolerated and mild to skin keratinocytes as determined by morphologic evaluation and MTS viability assessment. Surprisingly, fermented parsley exhibited dose dependent decreases in interleukin-6 (IL-6) and interleukin-8 (IL-8) cytokines that matched the 50 μg/ml benchmark dexamethasone when administered at a 10% concentration. Furthermore, lipopolysaccharide (LPS) stimulated inflammatory mediators were unexpectedly more significantly reduced using the fermented parsley rather than a water extract of parsley.

HaCaT Cell Culture: In an embodiment, HaCaT cells were cultured in Dulbecco's Modified Eagle Medium (DMEM), supplemented with 10% fetal bovine serum (FBS), and 1% penicillin (1×105 U/L)-streptomycin (100 mg/L). The cells were incubated at 37° C. in a humidified atmosphere with 5% CO₂. The culture medium was removed, and cells were digested using 0.25% trypsin (with ethylenediaminetetraacetic acid (EDTA)). Cells were then plated in 96-well culture plates for 12 hours, and then treated with different concentrations of the parsley ferment composition (ranging from 0.15625 to 40%) for 24 hours. Cell viability determination was accomplished using Cell Counting Kit-8 according to the manufacturer's protocol.

LPS Inflammatory Assay: In an embodiment, HaCaT cells were seeded in a 4 cm cell culture dish and incubated for 24 hours. Following incubation, 800 μg/ml of LPS was added to each dish excluding the control. At six hours post stimulation, LPS containing media was removed and cells were washed with phosphate-buffered saline (PBS) and replaced with 1 mL of growth media containing test materials or the positive control dexamethasone (50 ug/mL). Cells were incubated an additional 24 hours prior to collection of the media supernatant for measurement of inflammatory mediators. Cell monolayers were dissolved with 100 μL of cell lysis buffer, scraped and collected on ice, and lysate was used to detect the total protein content in the sample by BCA assay, following manufacturer's instructions. Quantification of various inflammatory factors in the cell supernatants was accomplished by enzyme-linked immunosorbent assay (ELISA), following manufacturer's instructions. Measured inflammatory mediators included IL-6 and IL-8.

The foregoing experiments were performed using at least triplicate samples, and data were expressed as mean ±standard deviation (SD). The data was analyzed using SPSS 17.0(SPSS, USA) and GraphPad Prism 9.0 (GraphPad Software, USA) software. One-way analysis of variance was used to determine the significance of differences between groups (*p<0.05; **p<0.01, ***p<0.001, n.s.p>0.05). Results and discussion follow.

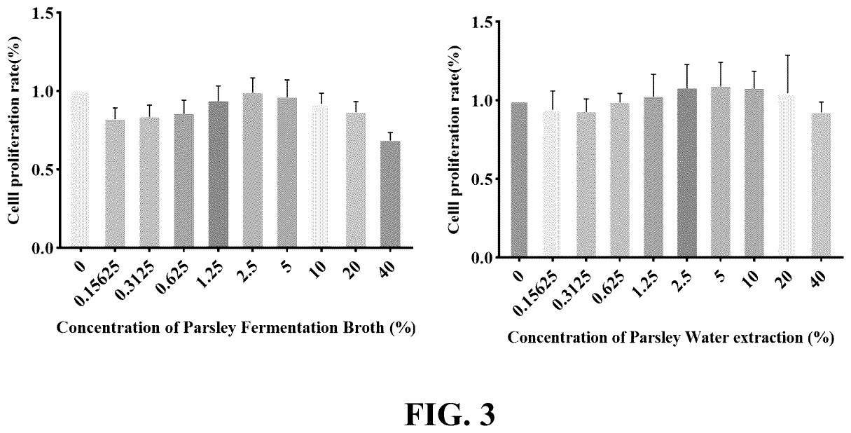

HaCaT Cell Viability Assay: With reference to FIG. 3, parsley fermented by a strain of Saccharomyces was used either neat following fermentation (Fermented Parsley Water) or combined with oils to create a broth (Fermented Parsley Broth). HaCaT cell viability was assessed following a 24-hour incubation with both ferment variants. Following a 24-hour incubation, cell viability and number was relatively unchanged. Slight decreases were observed at both the 20 and 40% concentrations. However, the cells' healthy morphologic appearance suggests that this effect is due to slowing of cellular division and not toxicity. Based on this data, concentrations of 2.5, 5 and 10% were chosen to perform inflammatory studies.

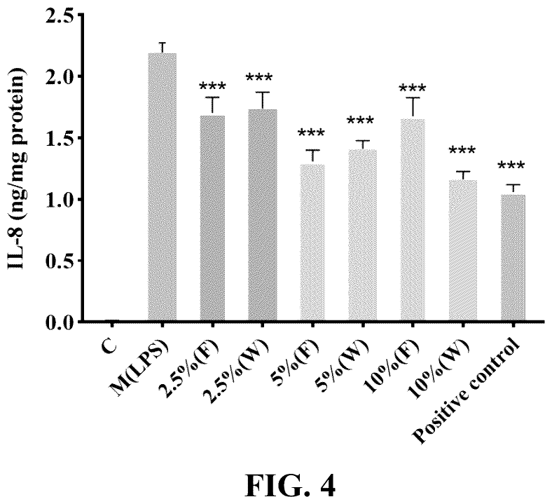

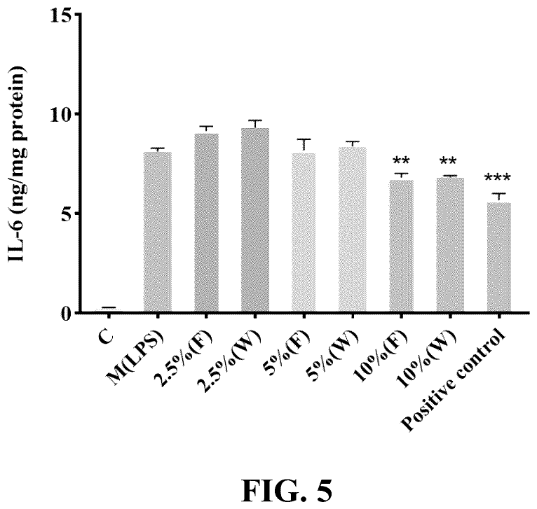

Inflammatory assay cytokine release profiles: With reference to FIGS. 4 and 5, HaCaT cell keratinocytes were used to assess the levels of inflammatory cytokines that were stimulated by LPS. Fermented parsley was used either neat following fermentation (W=Fermented Parsley Water), or combined with oils to create a broth (F=Fermented Parsley Broth). Dexamethasone was used as a positive control. Cytokines quantified included IL-8 (***p<0.001) and IL-6 (**p<0.01; ***p<0.001). The addition of LPS yielded a robust, statistically significant pro-inflammatory response for both cytokines measured. The positive control dexamethasone (50 μg/ml) significantly inhibited LPS stimulated release of both cytokines measured in this study.

Of the two fermented variants investigated, the capacity to inhibit both cytokines were nearly identical. Interestingly, the ferments were more robust at inhibiting IL-8 in this study. In both studies, 10% ferment was surprisingly as effective as the positive control dexamethasone at inhibiting inflammation, displaying statistically significant reductions for both. Specifically, the 10% Fermented Parsley Water unexpectedly and successfully inhibited LPS stimulated release of IL-8 to approximately 1.0 ng/mg protein. The 10% Fermented Parsley Water and 10% Fermented Parsley Broth inhibited LPS stimulated release of IL-6 to approximately 7.0 ng/mg protein.

While the present invention has been described through illustrative embodiments, routine modification will be apparent to those skilled in the art and such modifications are intended to be within the scope of this disclosure.

1-29. (canceled)

30. A cosmetic composition for treating inflammation comprising an extract of Carum Petroselinum (parsley), Ferment Filtrate, and 1,2 Hexanediol, Hydroxyacetophenone.

31. The cosmetic composition of claim 30, wherein the composition is for topical administration in a form of a lotion, cream, or gel.

32. The cosmetic composition of claim 30, wherein the composition is administered at a 2.5% concentration.

33. The cosmetic composition of claim 30, wherein the composition is administered at a 5% concentration.

34. The cosmetic composition of claim 30, wherein the composition is administered at a 10% concentration.

35. The cosmetic composition of claim 30, wherein the Ferment Filtrate comprises Saccharomyces Ferment Filtrate.

36. The cosmetic composition of claim 30, wherein the composition comprises 0.5-2.0% parsley extract, 96.0-99.0% Ferment Filtrate, 0.5-2.0% Hydroxyacetophenone, and 0.2-0.6% 1,2 Hexanediol by weight.

37. The cosmetic composition of claim 30, wherein the composition is administered neat as a water-based composition.

38. The cosmetic composition of claim 30, wherein the composition is combined with one or more oils and administered as a broth.

39. A method of treating inflammation, the method comprising administering to an individual in need of treatment a cosmetic composition comprising an extract of Carum Petroselinum (parsley), Ferment Filtrate, and 1,2 Hexanediol, Hydroxyacetophenone.

40. The method of claim 39, wherein administering the composition comprises topically administering the composition in a form of a lotion, cream, or gel to an individual in need of treatment.

41. The method of claim 39, wherein administering the composition comprises administering the composition at a 2.5% concentration.

42. The method of claim 39, wherein administering the composition comprises administering the composition at a 5% concentration.

43. The method of claim 39, wherein administering the composition comprises administering the composition at a 10% concentration.

44. The method of claim 39, wherein administering the composition comprises administering a composition comprising an extract of Carum Petroselinum (parsley), Saccharomyces Ferment Filtrate, and 1,2 Hexanediol, Hydroxyacetophenone.

45. The method of claim 39, wherein administering the composition comprises administering a composition that is 0.5-2.0% parsley extract, 96.0-99.0% Ferment Filtrate, 0.5-2.0% Hydroxyacetophenone, and 0.2-0.6% 1,2 Hexanediol by weight.

46. The method of claim 39, wherein administering the composition comprises administering the composition neat as a water-based composition.

47. The method of claim 39, wherein administering the composition comprises combining the composition with one or more oils and administering the composition as a broth.

48. A method of manufacturing a cosmetic composition for treating inflammation, the method comprising:
combining ingredients comprising an extract of Carum Petroselinum (parsley) with water and yeast;
fermenting the combined ingredients;
inactivating and decolorizing the fermented ingredients;
subjecting the composition to centrifugal filtration;
adding 1,2 Hexanediol, Hydroxyacetophenone to the composition;
and subjecting the composition to at least one round of sterilization.

49. The method of claim 48, wherein combining the extract of parsley with water and yeast comprises combining an extract of parsley with water and Saccharomyces.

50. The method of claim 48, wherein the composition comprises 0.5-2.0% parsley extract, 96.0-99.0% Ferment Filtrate, 0.5-2.0% Hydroxyacetophenone, and 0.2-0.6% 1,2 Hexanediol by weight.

51. The method of claim 48, wherein fermenting the combined ingredients comprises fermenting the combined ingredients at 28°C for 72 hours.

52. The method of claim 48, wherein the composition is manufactured for topical administration in a form of a lotion, cream, or gel.

53. A method for repairing inflammation damage in human keratinocytes, the method comprising applying to the keratinocytes a composition comprising a fermented Carum Petroselinum (parsley) extract.

54. The method of claim 53, wherein applying the composition comprises applying a composition comprising a Saccharomyces fermented Carum Petroselinum (parsley) extract.

55. The method of claim 53, wherein applying the composition comprises applying a composition comprising 0.5-2.0% parsley extract, 96.0-99.0% Ferment Filtrate, 0.5-2.0% Hydroxyacetophenone, and 0.2-0.6% 1,2 Hexanediol by weight.

56. The method of claim 53, wherein applying the composition comprises topically applying the composition to the keratinocytes in a form of a lotion, cream, or gel.

57. The method of claim 53, wherein applying the composition comprises applying the composition at a 2.5% concentration.

58. The method of claim 53, wherein applying the composition comprises applying the composition at a 5% concentration.

59. The method of claim 53, wherein applying the composition comprises applying the composition at a 10% concentration.

60. The method of claim 53, wherein applying the composition comprises applying the composition neat as a water-based composition.

61. The method of claim 53, wherein applying the composition comprises applying the composition as a broth.
